(19) United States
(12) Patent Application Publication
(10) Pub. No.: US 2024/0024614 A1
(43) Pub. Date: Jan. 25, 2024

(54) Awakening Inducting Method, Awakening Inducting Program, and Awakening Inducting System

(71) Applicant: Nippon Telegraph and Telephone Corporation, Tokyo (JP)

(72) Inventors: Daichi Matsunaga, Musashino-shi, Tokyo (JP); Yujiro Tanaka, Musashino-shi, Tokyo (JP); Takuro Tajima, Musashino-shi, Tokyo (JP); Michiko Seyama, Musashino-shi, Tokyo (JP)

(21) Appl. No.: 18/041,099

(22) PCT Filed: Sep. 2, 2020
(86) PCT No.: PCT/JP2020/033219

§ 371 Date: Feb. 9, 2023

(51) Int. Cl. A61M 21/00

(52) U.S. Cl. CPC A61M 21/00; A61M 2021/0044; A61M 2021/0083; A61M 2021/0066; A61M 2230/50

(57) ABSTRACT

An awakening induction system includes a core temperature measuring unit that measures a core temperature of a user; an awakening time estimation unit that estimates an awakening time of the user on the day before a scheduled date of awakening induction; a recommended wake-up time presentation unit that determines a recommended wake-up time range of the scheduled date based on the awakening time to present the wake-up time range to the user; a wake-up time setting unit that sets a scheduled wake-up time of the scheduled date according to an instruction from the user; and a control unit that instructs the environmental control device to raise an environment temperature around the user when it is a predetermined time before the scheduled wake-up time of the scheduled date, and also instructs the environmental control device to irradiate the user with light when the scheduled wake-up time of the scheduled date is reached.

### TECHNICAL FIELD

The present invention relates to an awakening induction method, an awakening induction program and an awakening induction system, each of which is able to induce a person to transition from sleep to awake.

### BACKGROUND ART

It is known that wakefulness of a person can be improved by light irradiation and adjustment of an environmental temperature (see NPL 1 and NPL 2). On the other hand, social jet lag caused by discrepancies between an individual's circadian rhythm and social clock is an issue for many people living in modern society. In a case where an individual is awakened by irradiating light or adjusting the environmental temperature as stated above, a heavy burden is placed on the mind and body because of the biological rhythm disruption.

### CITATION LIST

### Non Patent Literature

[NPL 1] Hiroko Sawai et al., “Impact of Practical Light Environment Control on Wakefulness and Performance: Comparison of the Impact of Gradient Increase in Illuminance between Illuminances and Light Sources,” AIJ, Information Systems Technical Committee, 35th Symposium on Computer Technology of Information, Systems and Applications 2012, R-19, pp. 97-102, 2012
[NPL 2] Kazuyo Tsuzuki, “Thermal Environment and Sleep”, Japanese Journal of Biometeorology, Vol. 50, No. 4, pp. 125-134, 2014

### SUMMARY OF INVENTION

### Technical Problem

The present invention is intended to solve the problem stated above, and its object is to provide an awakening induction method, an awakening induction program and an awakening induction system, each of which is capable of awakening a person without imposing a heavy burden on the mind and body.

### Solution to Problem

An awakening induction method of the present invention includes, a first step of measuring a core temperature of a user; a second step of estimating an awakening time of the user on the day before a scheduled date of awakening induction based on previous measurement of the core temperature, a third step of determining a recommended wake-up time range of the scheduled date based on the awakening time to present the wake-up time range to the user; a fourth step of setting a scheduled wake-up time of the scheduled date according to an instruction from the user; a fifth step of raising an environment temperature around the user when it is a predetermined time before the scheduled wake-up time of the scheduled date; and a sixth step of irradiating the user with light when the scheduled wake-up time of the scheduled date is reached.

An awakening induction program of the present invention is a program causing a computer to execute each step as described above.

An awakening induction system of the present invention includes an environmental control device configured to control an environment around a user; a core temperature measuring unit configured to measure a core temperature of the user; an awakening time estimation unit configured to estimate an awakening time of the user on the day before a scheduled date of awakening induction based on previous measurement of the core temperature; a recommended wake-up time presentation unit configured to determine a recommended wake-up time range of the scheduled date based on the awakening time to present the wake-up time range to the user; a wake-up time setting unit configured to set a scheduled wake-up time of the scheduled date according to an instruction from the user; and a control unit configured to instruct the environmental control device to raise an environment temperature around the user when it is a predetermined time before the scheduled wake-up time of the scheduled date, and to instruct the environmental control device to irradiate the user with light when the scheduled wake-up time of the scheduled date is reached.

### Advantageous Effects of Invention

According to the present invention, it is possible to awake a user without imposing a heavy burden on the mind and body by giving stimulation to the user at proper timing based on a core temperature of the user to induce them to transition from sleep to awake.

### BRIEF DESCRIPTION OF DRAWINGS

FIG. 1 is a block diagram illustrating a configuration of an awakening induction system according to an example of the present invention.

FIG. 2 is a flowchart illustrating operations of the awakening induction system according to the example of the present invention.

FIG. 3 is a diagram illustrating a method for estimating an awakening time in the example of the present invention.

FIG. 4 is a diagram illustrating an awakening induction method in the example of the present invention.

FIG. 5 is a block diagram illustrating an exemplified configuration of a computer which implements the awakening induction system according to the example of the present invention.

### DESCRIPTION OF EMBODIMENTS

### Principles of the Invention

A circadian rhythm, also known as a biological rhythm, repeats roughly every 24 hours. A core temperature strongly reflecting the circadian rhythm is higher during daytime, reducing from approximately one hour before sleeping, and lower during sleep. The core temperature rises from approximately one hour before wake-up. The circadian rhythm can be regulated by external stimuli such as light, but the regulation is limited to only several hours a day. The forcible regulation of the circadian rhythm leads to a heavy burden on the mind and body of a human subject. Therefore, the present invention is intended to induce a person to transition from sleep to awake by giving stimulation to the person at proper timing based on the core temperature.

### Examples

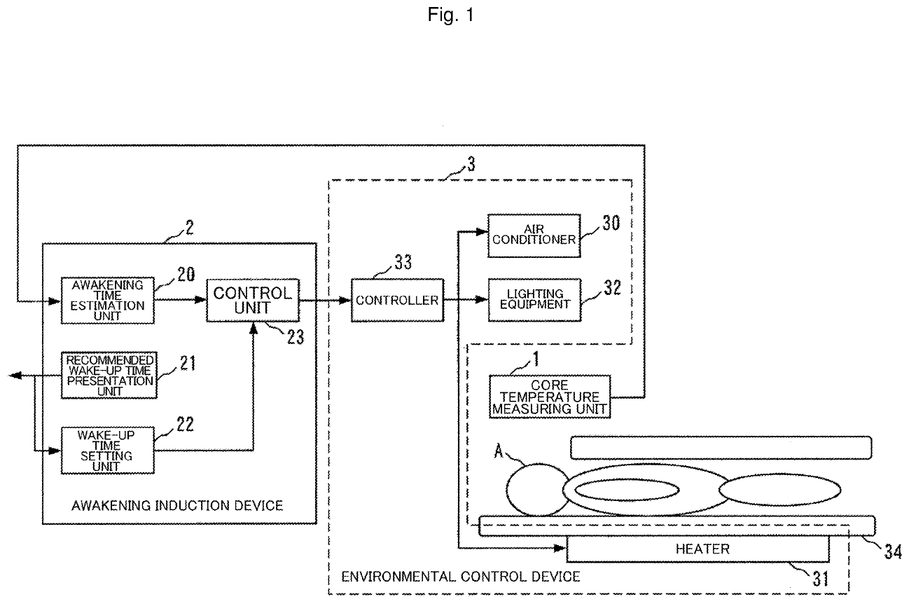

Referring to the drawings, the present invention will be described with examples. FIG. 1 is a block diagram illustrating a configuration of an awakening induction system according to an example of the present invention. The awakening induction system is configured by a core temperature measuring unit 1 that measures a core temperature of a user A, an awakening induction device 2, and an environmental control device 3 that controls an environment around the user A.

The awakening induction device 2 is configured by an awakening time estimation unit 20 that estimates an awakening time of the user A on the day before a scheduled date of awakening induction; a recommended wake-up time presentation unit 21 that determines a recommended wake-up time range of the scheduled date based on the awakening time to present the wake-up time range to the user A; a wake-up time setting unit 22 that sets a scheduled wake-up time of the scheduled date according to an instruction from the user; and a control unit 23 that instructs the environmental control device 3 to raise an environment temperature around the user A when it is at a predetermined time before the scheduled wake-up time of the scheduled date, and also instructs the environmental control device 3 to irradiate the user A with light when the scheduled wake-up time of the scheduled date is reached.

The environmental control device 3 is configured to an air conditioner 30 provided in a room where the user A sleeps, a heater 31 provided in a bedding used by the user A, a lighting equipment 32 provided in the room where the user A sleeps, and a controller 33 that controls the air conditioner 30, the heater 31 and the lighting equipment 32.

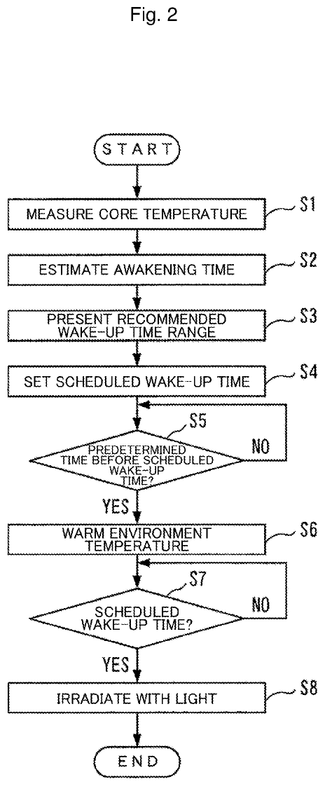

FIG. 2 is a flowchart illustrating operations of the awakening induction system according to the example of the present invention. The core temperature measuring unit 1 measures a core temperature Tc of the user A (step S1 in FIG. 2). A device for measuring the core temperature Tc from an eardrum temperature by mounting a sensor on, for example, the ear of the user A can be adopted as the core temperature measuring unit 1. However, in the present invention, the core temperature TC of the user A may be measured by another method. The core temperature Tc may be estimated from a dermal temperature. In order to estimate a wake-up time as described later, the core temperature measuring unit 1 needs to continuously measure the core temperature of the user A at least from when the user goes to sleep to when the user wakes up.

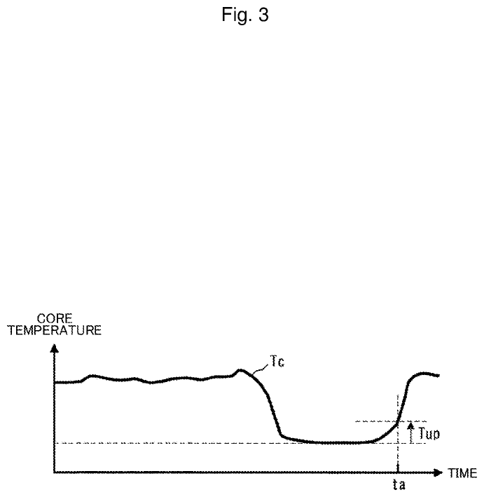

The user A who wakes up instructs the awakening induction device 2 to perform awakening induction for inducing the user to transition from sleep to awake on the next day. In response to the instruction from the user A, the awakening time estimation unit 20 estimates an awakening time ta of the user A today (on the day before a scheduled date of awakening induction) based on previous measurement of the core temperature Tc by the core temperature measuring unit 1 (step S2 in FIG. 2). More specifically, as shown in FIG. 3, the awakening time estimation unit 20 sets a time when the core temperature Tc rises by a specified temperature Tup (for example, 0.2 to 0.5° C.) from the latest lowest temperature Tmin as the awakening time ta of the user A today.

The recommended wake-up time presentation unit 21 determines a recommended wake-up time range R for the next day (the scheduled date of awakening induction) based on the calculated awakening time ta, and presents it to the user A (step S3 in FIG. 2). The recommended wake-up time presentation unit 21 sets a predetermined time range around the awakening time ta, for example, the awakening time ta±1 hour, as the recommended wake-up time range R.

The user A selects a desired time as a scheduled wake-up time tg for the next day in the presented wake-up time range R. The wake-up time setting unit 22 sets a scheduled wake-up time tg instructed by the user A for the control unit 23 (step S4 in FIG. 2).

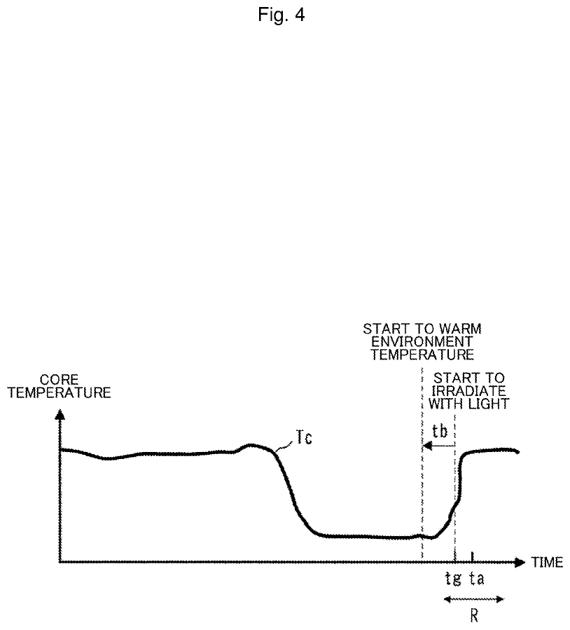

When it is a predetermined time tb (for example, 1 hour) before the scheduled wake-up time tg of the scheduled date (YES at step S5 in FIG. 2), the control unit 23 instructs the environmental control device 3, as shown in FIG. 4, to raise an environment temperature around the user A, thereby helping the user A raise the core temperature Tc (step S6 in FIG. 2).

In response to the instruction from the control unit 23, the controller 33 of the environmental control device 3 controls the air conditioner 30 provided in the room where the user A sleeps and the heater 31 provided in a bedding 34 used by the user A, thereby raising the environment temperature. The controller 33 may raise the environment temperature using at least one of the air conditioner 30 and the heater 31. Further, the controller 33 may control at least one of the air conditioner 30 and the heater 31 with a value obtained by, for example, adding a predetermined rise temperature to the environment temperature at the predetermined time tb before the scheduled wake-up time tg, as a control target value, such that the target value coincides with the environment temperature. Or alternatively, the controller 33 may control at least one of the air conditioner 30 and the heater 31 with a preset temperature as a control target value, such that the target value coincides with the environment temperature.

Subsequently, the control unit 23 confirms that the core temperature Tc of the user A, measured by the core temperature measuring unit 1, raises from the lowest temperature, and when the scheduled wake-up time tg is reached (YES at step S7 in FIG. 2), instructs the environmental control device 3 to irradiate the user A with light (step S8 in FIG. 2). In response to the instruction from the control unit 23, the controller 33 of the environmental control device 3 turns on the lighting equipment 32 provided in the room where the user A sleeps.

Thus, in this example, awakening induction can be performed at proper timing, and the user A can wake up without imposing a heavy burden on the mind and body of the user A.

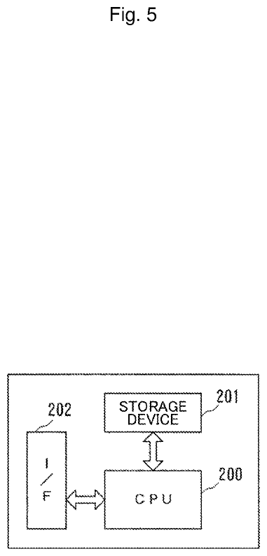

The awakening induction device 2 described in the present example can be implemented by a computer having a central processing unit (CPU), a storage device and an interface, and a program that controls these hardware resources. FIG. 5 illustrates an exemplified configuration of the computer.

The computer includes a CPU 200, a storage device 201, and an interface device (I/F) 202. The core temperature measuring unit 1 and the controller 33 of the environmental control device 3 are connected to the I/F 202. A program for implementing the awakening induction method of the present invention is stored in the storage device 201 of the computer. The CPU 200 executes the processing described in the present example according to the program stored in the storage device 201. The controller 33 of the environmental control device 3 can also be implemented by the computer.

### INDUSTRIAL APPLICABILITY

The present invention can be applied to techniques for awakening a person.

### REFERENCE SIGNS LIST

1 Core temperature measuring unit
2 Awakening induction device
3 Environmental control device
20 Awakening time estimation unit
21 Wake-up time presentation unit
22 Wake-up time setting unit
23 Control unit
30 Air conditioner
31 Heater
32 Lighting equipment
33 Controller

1. An awakening induction method, comprising:
a first step of measuring a core temperature of a user;
a second step of estimating an awakening time of the user on the day before a scheduled date of awakening induction based on previous measurement of the core temperature,
a third step of determining a recommended wake-up time range of the scheduled date based on the awakening time to present the wake-up time range to the user;
a fourth step of setting a scheduled wake-up time of the scheduled date according to an instruction from the user;
a fifth step of raising an environment temperature around the user when it is a predetermined time before the scheduled wake-up time of the scheduled date; and
a sixth step of irradiating the user with light when the scheduled wake-up time of the scheduled date is reached.

2. The awakening induction method according to claim 1, wherein the second step includes a step of setting, as the awakening time, a time when the core temperature rises from the latest lowest temperature by a predetermined temperature upon estimating the awakening time on the day before the scheduled date.

3. The awakening induction method according to claim 1, wherein the third step includes a step of setting a predetermined time range around the awakening time as the recommended wake-up time range.

4. The awakening induction method according to claim 1, wherein the fifth step includes a step of controlling at least one of an air conditioner provided in a room where the user sleeps and a heater provided in a bedding used by the user to increase an environmental temperature around the user.

5. An awakening induction program causing a computer to execute each step as set forth in claim 1.

6. An awakening induction system, comprising:
an environmental control device configured to control an environment around a user;
a core temperature measuring unit configured to measure a core temperature of the user;
an awakening time estimation unit configured to estimate an awakening time of the user on the day before a scheduled date of awakening induction based on previous measurement of the core temperature;
a recommended wake-up time presentation unit configured to determine a recommended wake-up time range of the scheduled date based on the awakening time to present the wake-up time range to the user;
a wake-up time setting unit configured to set a scheduled wake-up time of the scheduled date according to an instruction from the user; and
a control unit configured to instruct the environmental control device to raise an environment temperature around the user when it is a predetermined time before the scheduled wake-up time of the scheduled date, and to instruct the environmental control device to irradiate the user with light when the scheduled wake-up time of the scheduled date is reached.

7. The awakening induction method according to claim 2, wherein the third step includes a step of setting a predetermined time range around the awakening time as the recommended wake-up time range.

8. The awakening induction method according to claim 2, wherein the fifth step includes a step of controlling at least one of an air conditioner provided in a room where the user sleeps and a heater provided in a bedding used by the user to increase an environmental temperature around the user.

9. The awakening induction method according to claim 3, wherein the fifth step includes a step of controlling at least one of an air conditioner provided in a room where the user sleeps and a heater provided in a bedding used by the user to increase an environmental temperature around the user.

10. An awakening induction program causing a computer to execute each step as set forth in claim 2.

11. An awakening induction program causing a computer to execute each step as set forth in claim 3.

12. An awakening induction program causing a computer to execute each step as set forth in claim 4.
